(19) United States
(12) Patent Application Publication
(10) Pub. No.: US 2007/0255054 A1
(43) Pub. Date: Nov. 1, 2007

(54) OLIGONUCLEOTIDE SYNTHESIS WITH INTERMITTENT AND POST SYNTHETIC OXIDATION

(75) Inventors: Robert G. Kuimelis, Palo Alto, CA (US); Paul A. Bury, Thornton, CO (US)

Correspondence Address: AFFYMETRIX, INC;ATTN: CHIEF IP COUNSEL, LEGAL DEPT., 3420 CENTRAL EXPRESSWAY, SANTA CLARA, CA, 95051, US

(73) Assignee: Affymetrix, INC., Santa Clara, CA (US)

(21) Appl. No.: 11/619,046

(22) Filed: Jan. 2, 2007

Related U.S. Application Data
(60) Provisional application No. 60/754,666, filed on Dec. 30, 2005.

(51) Int. Cl. C07H 21/04

(52) U.S. Cl. 536/25.31

(57) ABSTRACT

Process for fabricating an array of polymers on a solid support is provided having the steps of coupling a monomer with a reactive side masked by a protecting group to a growing polymer chain, optionally capping the monomer or monomer chains that have not reacted in a given cycle, deprotecting the product with a deprotecting agent, repeating the above steps on regions of the support as necessary to give polymers having a desired sequence and length and oxidizing the inter monomer linkage post-synthetically.

### TECHNICAL FIELD

The present invention relates generally to polymer chemistry and to the solid phase chemical synthesis of polymers. More particularly, the invention relates to an improved method for synthesizing oligonucleotides wherein oxidation is performed post-synthetically or intermittently as opposed to at each monomer addition cycle. The invention has utility in the fields of biochemistry, molecular biology and pharmacology, and in medical diagnostic and screening technologies.

### BACKGROUND OF THE INVENTION

Solid phase chemical synthesis of DNA fragments is routinely performed using protected nucleoside phosphoramidites. S. L. Beaucage et al. (1981) Tetrahedron Lett. 22:1859. In this approach, the 3′-hydroxyl group of an initial 5′-protected nucleoside is first covalently attached to the polymer support. R. C. Pless et al. (1975) Nucleic Acids Res. 2:773 (1975). Synthesis of the oligonucleotide then proceeds by deprotection of the 5′-hydroxyl group of the attached nucleoside, followed by coupling of an incoming nucleoside-3′-phosphoramidite to the deprotected hydroxyl group. M. D. Matteucci et al. (1981) J. Am. Chem. Soc. 103:3185. The resulting phosphite triester is finally oxidized to a phosphorotriester to complete the internucleotide bond. R. L. Letsinger et al. (1976) J. Am. Chem. Soc. 98:3655. The steps of deprotection, coupling and oxidation are repeated until an oligonucleotide of the desired length and sequence is obtained.

The chemical group conventionally used for the protection of nucleoside 5′-hydroxyls is dimethoxytrityl (“DMT”), which is removable with acid. H. G. Khorana (1968) Pure Appl. Chem. 17:349; M. Smith et al. (1962) J. Am. Chem. Soc. 84:430. This acid-labile protecting group provides a number of advantages for working with both nucleosides and oligonucleotides. For example, the DMT group can be introduced onto a nucleoside regioselectively and in high yield. E. I. Brown et al. (1979) Methods in Enzymol. 68:109. Also, the lipophilicity of the DMT group greatly increases the solubility of nucleosides in organic solvents, and the carbocation resulting from acidic deprotection gives a strong chromophore, which can be used to indirectly monitor coupling efficiency. M. D. Matteucci et al. (1980) Tetrahedron Lett. 21:719. In addition, the hydrophobicity of the group can be used to aid separation on reverse-phase HPLC. C. Becker et al. (1985) J. Chromatogr. 326:219.

However, use of DMT as a hydroxyl-protecting group in oligonucleotide synthesis is also problematic. The N-glycosidic linkages of oligodeoxyribonucleotides are susceptible to acid catalyzed cleavage (N. K. Kochetkov et al., Organic Chemistry of Nucleic Acids (New York: Plenum Press, 1972)), and even when the protocol is optimized, recurrent removal of the DMT group with acid during oligonucleotide synthesis results in depurination. H. Shaller et al. (1963) J. Am. Chem. Soc. 85:3821. The N-6-benzoyl-protected deoxyadenosine nucleotide is especially susceptible to glycosidic cleavage, resulting in a substantially reduced yield of the final oligonucleotide. J. W. Efcavitch et al. (1985) Nucleosides & Nucleotides 4:267.

As various well known methods for chemical removal of DMT protecting groups involving application of acidic conditions resulted in undesired side reactions, reagents and methods were developed for light-directed synthesis of DNA probes, utilizing phosphoramidite monomers having photolabile or photoremovable protecting groups. The conventional approaches to manufacturing DNA probe arrays via photolabile protecting groups involve the basic steps of (1) monomer coupling (2) capping (3) oxidation (4) exposure. These steps follow the conventional phosphoramidite-based schemes originally established to synthesize DNA oligomers on solid supports using DMT-protected nucleoside phosphoramidite monomers in conjunction with trichloroacetic acid solutions for DMT removal. For a variety of reasons, it is advantageous to (1) reduce synthesis cycle-times and therefore increase probe array manufacturing throughput, and (2) minimize chemical exposure to increase probe fidelity. With high-efficiency photogroups (e.g., NNPOC, DEACMOC, NPPOC, etc.) the importance of the capping step is minimized. In addition, because photogroups do not rely on acidic reagents for deprotection, a condition that is incompatible with phosphite triester functionalities, it might be possible to perform the oxidation step in a post-synthesis manner as opposed to a step-wise manner. Such a modification to the synthesis steps would result in a significantly reduced manufacturing time for probe arrays, and would also be beneficial for long-mer synthesis. Meaningful long-mer synthesis is particularly challenging because of the accumulation of deleterious effects of minor side reactions, at least some of which correlate with chemical exposure time.

### SUMMARY OF THE INVENTION

The present invention discloses methods for fabricating arrays of polymers on a solid support. One disclosed method has the steps of (a) coupling a monomer with a reactive site masked by a protecting group to a growing polymer chain; (b) optionally capping the monomer or monomer chains that have not reacted in a giving cycle; (c) deprotecting the product with a deprotecting agent to expose the reactive group; (e) repeating steps (a)-(c) on regions of support as necessary to give a polymer having a desired sequence and length; and (f) oxidizing the intermonomer linkages post-synthetically to obtain the desired array. In preferred embodiments of the disclosed invention, the array of polymers is an array of nucleic acids or an array of oligonucleotides.

In the present invention, the monomer in the process is preferably a nucleotide. It is also disclosed that a nucleotide is preferably protected with a photolabile protecting group at its 5′ or 3′ hydroxyl moiety in accordance with the present invention.

In still other embodiments of the present invention, the present invention provides a process for fabricating an array of monomer on a solid support, comprising the steps of: (a) coupling a monomer with a reactive group masked by a protecting group to a growing polymer chain; (b) optionally capping the monomer or monomer chains that have not reacted in a giving cycle; (c) deprotecting the product with a deprotecting agent to expose the reactive site; (d) repeating steps (a)-(c) on regions of support as necessary to give a polymer having a desired sequence and length; and (f) oxidizing the intermonomer linkages intermittently at every N cycles to obtain the desired array, wherein N is about 5, 10, 25, 100 or more. It should be understood that prior to oxidation, the process employs additional steps of reacting the exposed reactive group with a protected monomer. The present invention discloses that these steps may be further repeated until the desired polymer array is fabricated. The present invention discloses that the array is preferably comprised of a polymer of between about 20 to about 100 monomers in length.

### BRIEF DESCRIPTION OF THE FIGURES

FIG. 1 is a schematic representation of the steps of oligonucleotide synthesis using the post synthesis and intermittent oxidation methods.

FIG. 2 is representative of the steps in a minimized post-synthesis cycle.

FIG. 3 illustrates the reverse phase High Pressure Liquid Chromatography (HPLC) traces comparing various synthesis cycles.

FIG. 4 illustrates the reverse phase HPLC traces of the phosphoramidite coupling reaction set forth after single and double post-synthesis oxidation.

FIG. 5 illustrates the reverse phase HPLC traces of 20-mer probes synthesized by the post-synthesis oxidation scheme and the intermittent oxidation scheme.

FIG. 6 illustrates the hybridization signal of 20-mer arrays fabricated by intermittent (N=10) and post-synthetic oxidation methods.

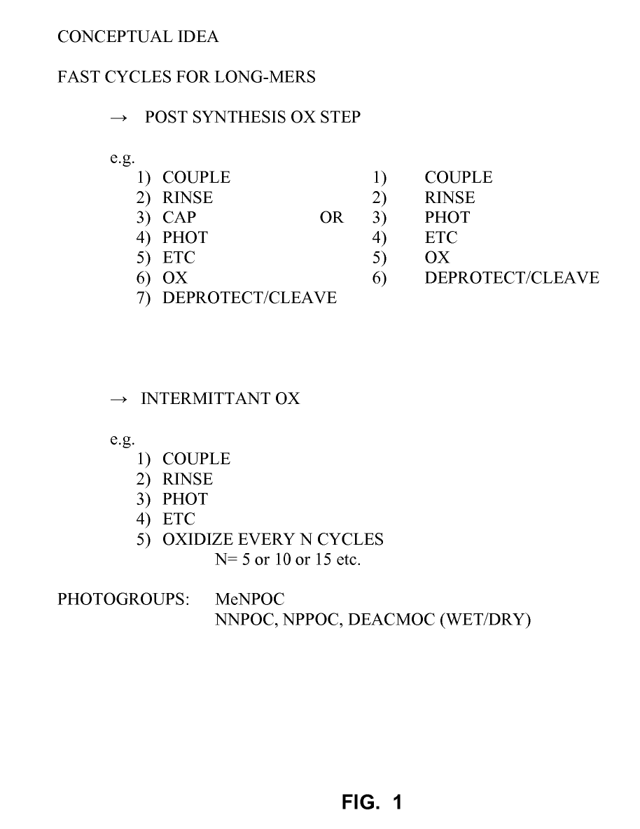

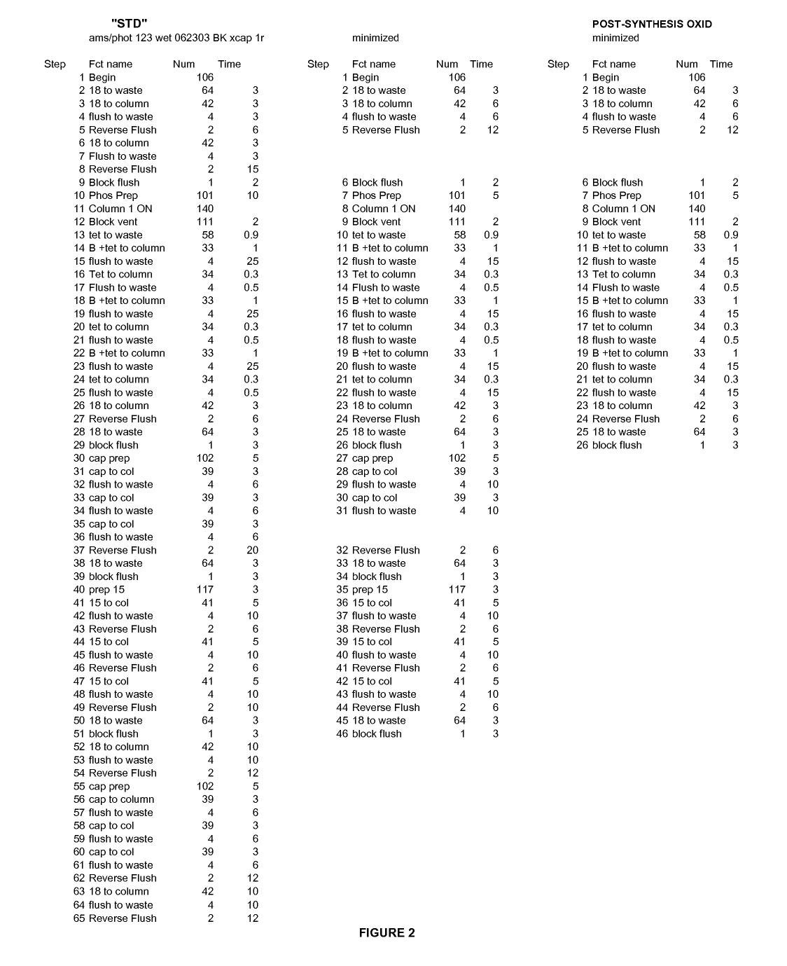

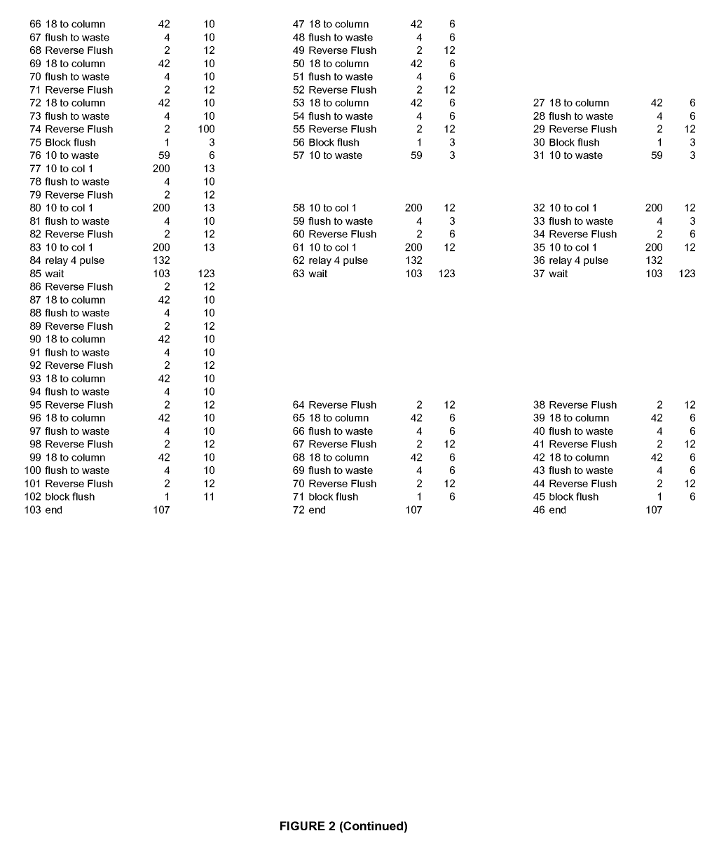

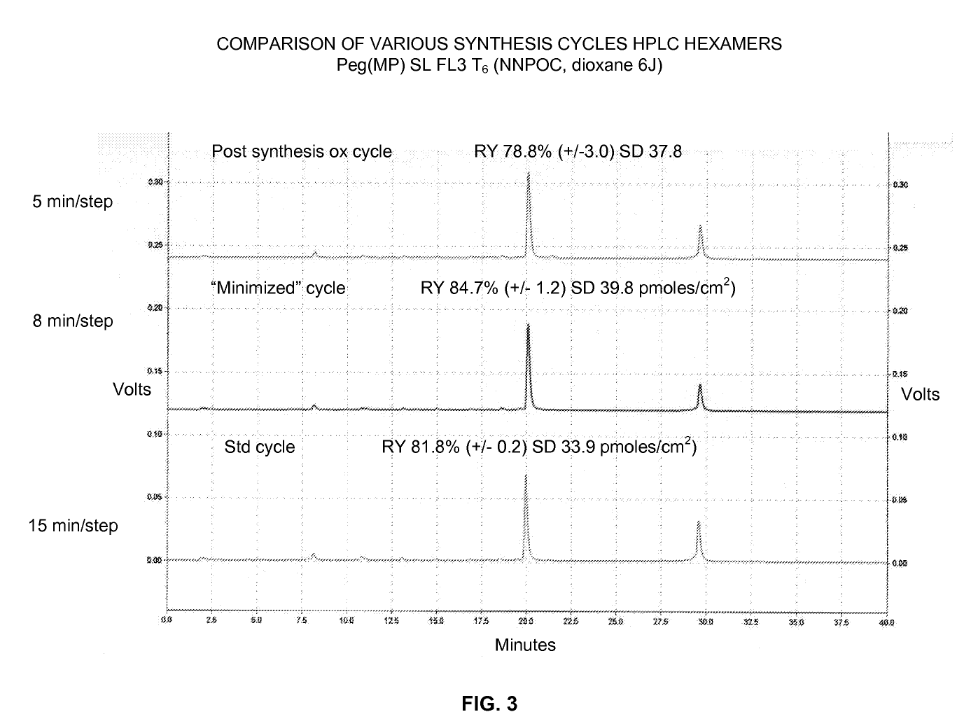

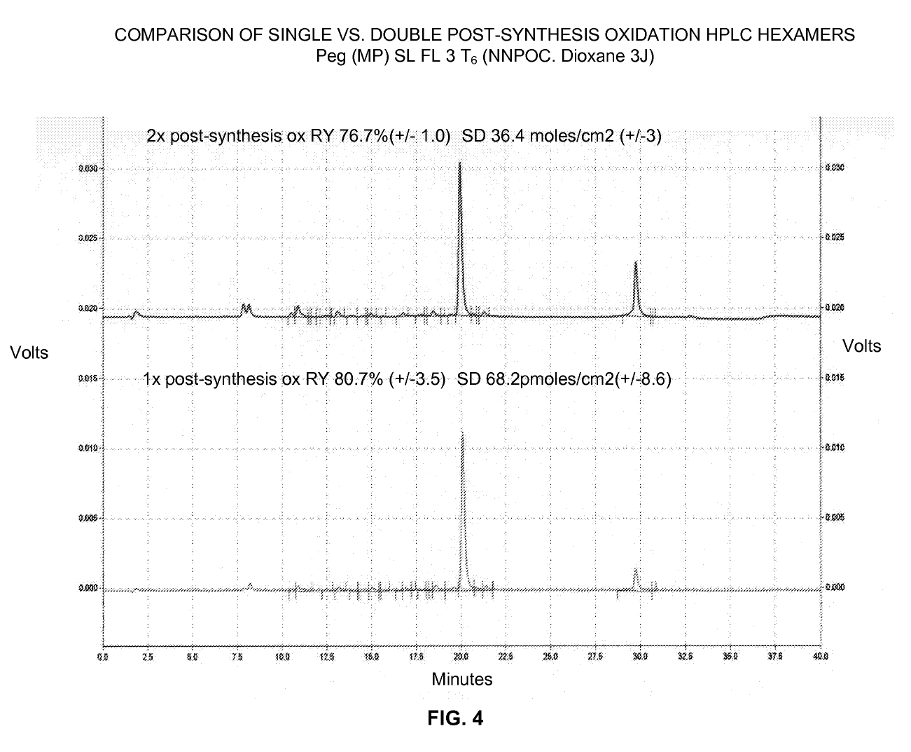

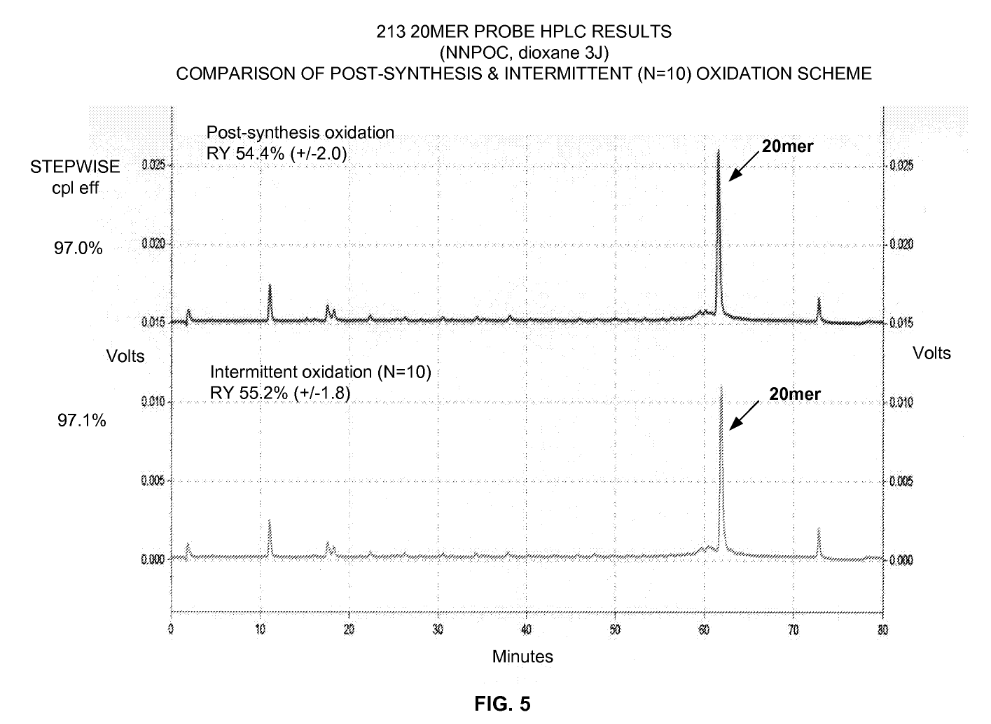

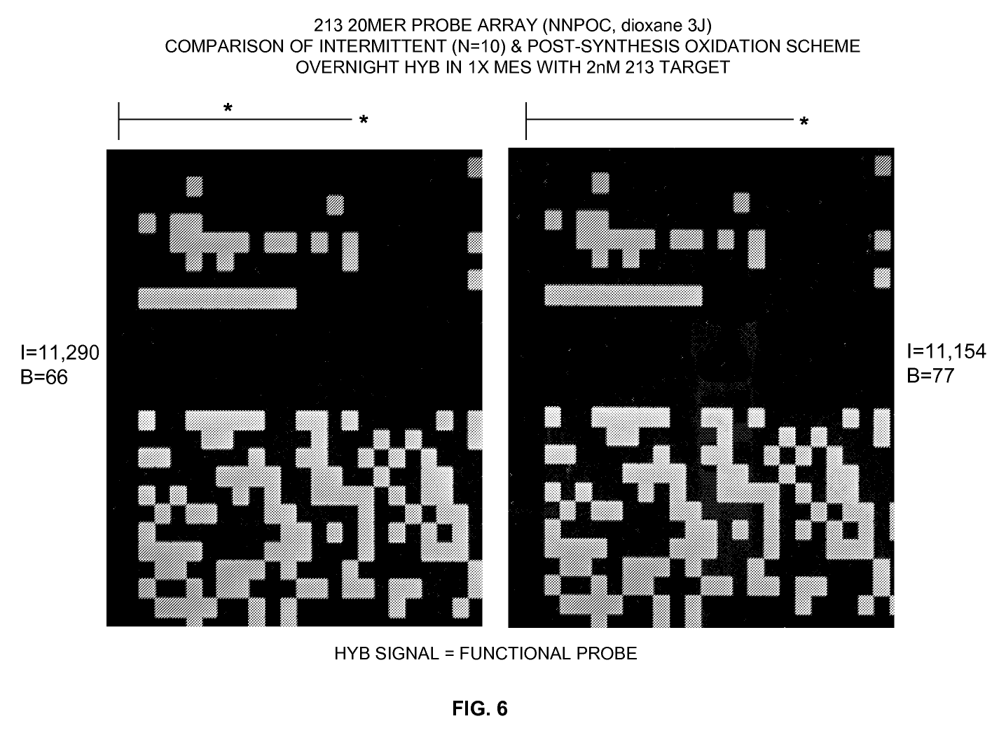

### DETAILED DESCRIPTION OF THE INVENTION

Definitions

As used herein, the following terms are intended to have the following general meanings:

Monomer: A monomer is a member of the set of small molecules which are or can be joined together to form a polymer or a compound composed of two or more members. The set of monomers includes but is not restricted to, for example, the set of common L-amino acids, the set of D-amino acids, the set of synthetic and/or natural amino acids, the set of nucleotides, and the set of pentoses and hexoses, each set of which is readily known to those of skill in the art. The particular ordering of monomers within a polymer is referred to herein as the “sequence” of the polymer. As used herein, “monomers” refers to any member of a basis set for synthesis of a polymer, and is not limited to a single “mer”. For example, dimers of the 20 naturally occurring L-amino acids form a basis set of 400 monomers for synthesis of polypeptides. Monomers can also include trimers, oligomers, polymers and so forth. Different basis sets of monomers may be used at successive steps in the synthesis of a polymer. Furthermore, each of the sets may include protected members composed of, protected amino acids as described above. Other examples of polymers within the scope of the present invention include without limitation linear and cyclic polymers of nucleic acids, polysaccharides, phospholipids, and peptides having either α-, β-, or ω-amino acids, heteropolymers in which a known drug is covalently bound to any of the above, polynucleotides, polyurethanes, polyesters, polycarbonates, polyamides, polyethyleneimines, polyarylene sulfides, polysiloxanes, polyimides, polyacetates, or other polymers within the scope of the present invention as would be understood by a person of kill in the art of this disclosure. Such polymers are “diverse” when polymers having different monomer sequences are formed at different predefined regions of a substrate. Methods of cyclization and polymer reversal are disclosed in copending application Ser. No. 796,727, filed Nov. 22, 1991, entitled “POLYMER REVERSAL ON SOLID SURFACES,” incorporated herein by reference in its entirety.

Nucleic Acids: Nucleic acids may be synthesized to establish sequences recognized by various receptor molecules, such as protein or other DNA or RNA molecules. Nucleic acids within the scope of the present invention include naturally occurring or synthetic bases or monomers, nucleic acid analogs, modified nucleic acids, nucleic acids containing modified nucleotides, modified nucleic acid analogs, oligonucleotides of whatever length, peptide nucleic acids and the like or mixtures thereof.

Substrate: A material having a rigid, semi-rigid or gelatinous surface. Typical examples include glass or suitable polymer materials. In some embodiments of the present invention, at least one surface of the substrate will be substantially flat, although in some embodiments it may be desirable to physically separate synthesis regions for different polymers with, for example, wells, raised regions, etched trenches, or the like. In some embodiments, the substrate itself contains wells, trenches, flow through regions, etc., which form all or part of the synthesis regions. According to other embodiments, small beads may be provided on the surface, and compounds synthesized thereon optionally may be released upon completion of the synthesis. Accordingly, the beads may be released after synthesis or the surface may comprise an array of bound beads or sets of released beads held with wells, trenches or other features on a surface. Substrates are well known in the art and are readily commercially available through vendors such as USPG, PPG Industries, AFG Industries and others.

Protective Group: A group or moiety which may be selectively removed to expose an active site such as an amino functionality in peptide or amino acid or a hydroxyl group in a nucleic acid or nucleotide. In accordance with one aspect of the present invention, protective groups may be removed under a variety of conditions, for example, depending on the nature of the protective group and the mode of its connection to the active sites basic or acidic conditions may be employed as appropriate. For an extensive listing of protective groups useful in the practice of the present invention, see also Greene, T. W. and Wuts, P. G. M., Protective Groups in Organic Synthesis, (1991), incorporated herein by reference in its entirety. Useful representative acid sensitive protective groups include dimethoxytrityl (DMT), tert-butylcarbamate (tBoc) and trifluoroacetyl (tFA). Useful representative base sensitive protective groups include 9-fluorenylmethoxycarbonyl (Fmoc), isobutyrl (iBu), benzoyl (Bz) and phenoxyacetyl (pac). Other protective groups include acetamidomethyl, acetyl, tert-amyloxycarbonyl, benzyl, benzyloxycarbonyl, 2-(4-biphenylyl)-2-propyloxycarbonyl, 2-bromobenzyloxycarbonyl, tert-butyl, tert-butyloxycarbonyl, 1-carbobenzoxamido-2,2,2-trifluoroethyl-1,2,6-dichlorobenzyl, 2-(3,5-dimethoxyphenyl)-2-propyloxycarbonyl, 2,4-dinitrophenyl, dithiasuccinyl, formyl, 4-methoxybenzenesulfonyl, 4-methoxybenzyl, 4-methylbenzyl, o-nitrophenylsulfenyl, 2-phenyl-2-propyloxycarbonyl, .alpha.-2,4,5-tetramethylbenzyloxycarbonyl, p-toluenesulfonyl, xanthenyl, benzyl ester, N-hydroxysuccinimide ester, p-nitrobenzyl ester, p-nitrophenyl ester, phenyl ester, p-nitrocarbonate, p-nitrobenzylcarbonate, trimethylsilyl and pentachlorophenyl ester and the like.

Predefined Region: A predefined region is a localized area on a substrate which is, was, or is intended to be used for formation of a selected polymer and is otherwise referred to herein in the alternative as “reaction” region, a “selected” region, simply a “region” or a feature. The predefined region may have any convenient shape, e.g., circular, rectangular, elliptical, wedge-shaped, etc. In accordance with the present invention, the arrays of the present invention have features on the order of 10-100 μm, i.e., 10×10 μm² to 100×100 μm² for approximately square features. More preferably the features will be on the order of 1-10 μm. It is also an object of the present invention to provide features having sub-micron dimensions. Such features are preferably on the order of 100-1000 nm. Within these regions, the polymer synthesized therein is preferably synthesized in a substantially pure form. However, in other embodiments of the invention, predefined regions may substantially overlap. In such embodiments, hybridization results may be resolved by software for example.

A Deprotecting Agent is a chemical or agent which causes a Protective Group to be cleaved from, for example, a protected monomer. Such cleavage, in accordance with the present invention, preferably exposes a reactive group on the monomer. The reactive group may then, in accordance with the present invention, be used to couple the deprotected monomer to the next monomer creating the polymer step by step using the appropriate chemistry. This next monomer coupled would, in accordance with one aspect of the present invention, bear a protective group which could in turn be cleaved under appropriate conditions.

An Activatable Deprotecting Agent is a chemical or agent which is relatively inert with respect to a Protective Group bound to a monomer, i.e., the activatable deprotecting agent will not cause cleavage of the protective group in any significant amount absent activation. An activatable deprotecting agent may be activated in a variety of ways depending on its chemical and physical properties. In accordance with one aspect of the present invention, certain activatable deprotecting agents may be activated by exposure to some form of activator, e.g. electromagnetic radiation. In accordance with one aspect of the present invention, an activatable deprotecting agent will be activatable at only certain wave lengths of electromagnetic radiation and not at others. For example, certain activatable deprotecting reagents will be activated with visible or UV light.

Solid support refers to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations.

Isolation and purification of the compounds and intermediates described herein can be effected, if desired, by any suitable separation or purification procedure such as, for example, filtration, extraction, crystallization, column chromatography such as HPLC, thin-layer chromatography, thick-layer (preparative) chromatography, distillation, or a combination of these procedures. Specific illustrations of suitable separation and isolation procedures can be had by references to the examples hereinbelow. However, other equivalent separation or isolation procedures can, or course, also be used.

The present invention has many preferred embodiments and relies on many patents, applications and other references for details known to those of skill in the art. Therefore, when a patent, application, or other reference is cited or repeated below, it is incorporated by reference in its entirety unless indicated otherwise.

As used in this application, the singular form “a,” “an,” and “the” include the corresponding plural references unless the context dictates otherwise. Likewise, plural references include the singular unless the context indicates otherwise.

Throughout this disclosure, various aspects of this invention can be presented in a range format. It should be understood that such description is merely for convenience and brevity and should not be construed as an unwarranted limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques of organic chemistry, polymer technology, molecular biology (including recombinant nucleic acid techniques), cell biology, biochemistry, and immunology as would be understood by one of the ordinary skill. Such conventional techniques include polymer array synthesis, hybridization, ligation, and detection of hybridization using a label. Specific illustrations of suitable techniques can be had by reference to the examples herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3^{rd} Ed., W.H. Freeman Pub., New York, N.Y. and Berg et al. (2002) Biochemistry, 5^{th} Ed., W.H. Freeman Pub., New York, N.Y., all of which are herein incorporated by reference in their entirety.

The present invention can employ solid substrates, including arrays in some preferred embodiments. Methods and techniques applicable to polymer (including protein) array synthesis have been described in U.S. Ser. No. 09/536,841, WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication Number WO 99/36760) and PCT/US01/04285 (International Publication Number WO 01/58593), which are all incorporated herein by reference in their entirety.

Patents that describe synthesis techniques in specific embodiments include U.S. Pat. Nos. 5,412,087, 6,147,205, 6,262,216, 6,310,189, 5,889,165, and 5,959,098, which are all incorporated by reference in their entirety. Nucleic acid arrays are described in many of the above patents, but the same general methodologies are applicable to polypeptide arrays.

The present invention also contemplates many uses for polymers attached to solid substrates. These uses include gene expression monitoring, profiling, library screening, genotyping and diagnostics. Gene expression monitoring, and profiling methods can be shown in U.S. Pat. Nos. 5,800,992, 6,013,449, 6,020,135, 6,033,860, 6,040,138, 6,177,248 and 6,309,822, which are all incorporated by reference in their entirety. Genotyping and uses therefore are shown in U.S. Ser. Nos. 60/319,253, 10/013,598 (U.S. Patent Application Publication 20030036069), and U.S. Pat. Nos. 5,856,092, 6,300,063, 5,858,659, 6,284,460, 6,361,947, 6,368,799 and 6,333,179, which are incorporated by reference in their entirety. Other uses are embodied in U.S. Pat. Nos. 5,871,928, 5,902,723, 6,045,996, 5,541,061, and 6,197,506, which are incorporated by reference in their entirety.

The present invention also contemplates sample preparation methods in certain preferred embodiments. Prior to or concurrent with genotyping, the genomic sample may be amplified by a variety of mechanisms, some of which may employ PCR. See, e.g., PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188, and 5,333,675, and each of which is incorporated herein by reference in their entirety. The sample may be amplified on the array. See, for example, U.S. Pat. No. 6,300,070 and U.S. Ser. No. 09/513,300, which are incorporated herein by reference in their entirety.

Other suitable amplification methods include the ligase chain reaction (LCR) (e.g., Wu and Wallace, Genomics 4, 560 (1989), Landegren et al., Science 241, 1077 (1988) and Barringer et al. Gene 89: 117 (1990)), transcription amplification (Kwoh et al., Proc. Natl. Acad. Sci. USA 86, 1173 (1989) and WO88/10315), self-sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5,413,909, 5,861,245) and nucleic acid based sequence amplification (NABSA). (See, U.S. Pat. Nos. 5,409,818, 5,554,517, and 6,063,603, each of which is incorporated herein by reference). Other amplification methods that may be used are described in, U.S. Pat. Nos. 5,242,794, 5,494,810, 4,988,617 and in U.S. Ser. No. 09/854,317. Each of the above references is incorporated herein by reference in its entirety.

Additional methods of sample preparation and techniques for reducing the complexity of a nucleic sample are described in Dong et al., Genome Research 11, 1418 (2001), in U.S. Pat. Nos. 6,361,947, 6,391,592 and U.S. Ser. Nos. 09/916,135, 09/920,491 (U.S. Patent Application Publication 20030096235), 09/910,292 (U.S. Patent Application Publication 20030082543), and 10/013,598, each of which is incorporated herein by reference in its entirety.

Numerous methods for conducting polynucleotide hybridization assays have been well developed. Hybridization assay procedures and conditions will vary depending on the application and are selected in accordance with the general binding methods known including those referred to in: Maniatis et al. Molecular Cloning: A Laboratory Manual (2nd Ed. Cold Spring Harbor, N.Y, 1989); Berger and Kimmel Methods in Enzymology, Vol. 152, Guide to Molecular Cloning Techniques (Academic Press, Inc., San Diego, Calif., 1987); Young and Davism, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623, each of which is hereby incorporated by reference in its entirety.

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. Ser. No. 60/364,731 and in PCT Application PCT/US99/06097 (published as WO99/47964), each of which also is hereby incorporated by reference in its entirety.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, e.g. Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2^{nd}, 2001). See U.S. Pat. No. 6,420,108. Each of these references is incorporated herein by reference in its entirety.

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170. Each of these references is incorporated herein by reference in its entirety.

Light patterns can also be generated using Digital Micromirrors, Light Crystal on Silicon (LCOS), light valve arrays, laser beam patterns and other devices suitable for direct-write photolithography. See. e.g., U.S. Pat. Nos. 6,271,957 and 6,480,324, incorporated herein by reference.

Additionally, the present invention may have preferred embodiments that include methods for providing genetic information over networks such as the Internet as shown in U.S. Ser. Nos. 10/063,559 (United States Publication No. U.S. 20020183936), U.S. Provisional Application 60/349,546, 60/376,003, 60/394,574 and 60/403,381). Each of these references is incorporated herein by reference in its entirety.

The present invention provides methods for the formation of arrays of large numbers of different polymer sequences. The invention also includes the arrays formed using the methods disclosed herein. One aspect of the invention includes methods for the synthesis of an array of different polymers in selected and predefined regions of a substrate. Another aspect of the invention includes those arrays and various methods of using them.

Such arrays are used in, for example, in nucleic acid analysis. Polynucleotide or nucleic acid arrays are especially suitable for checking the accuracy of previously elucidated sequences and for detecting mutations and polymorphisms. Polymer arrays are also used in screening studies to evaluate their interaction with, for example nucleic acids in the case, for example of oligonucleotide arrays. Nucleic acid sequences can also be immobilized in specific locations or predefined regions of a substrate using the current invention. In some embodiments, such immobilized nucleic acid arrays are used in hybridization assays for gene expression monitoring, nucleic acid amplifications, nucleic acid computation, and nucleic acid analysis in general.

The present invention has certain features in common with the radiation directed methods discussed in U.S. Pat. No. 5,143,854, incorporated herein by reference. The radiation-directed methods discussed in that patent involve activating predefined regions of the substrate and then contacting the substrate with a preselected monomer solution. The predefined regions can be activated with, for example, a light source shown through a mask (much in the manner of photolithographic techniques used in integrated circuit fabrication). Other regions of the substrate remain inactive because they are blocked by the mask from illumination. Thus, a light pattern defines which regions of the substrate react with a given monomer. By repeatedly activating different sets of predefined regions and providing different monomer compositions thereto, a diverse array of polymers is produced on or near the substrate.

In one preferred embodiment, the monomer is a 2′-deoxynucleoside phosphoramidite containing an acid removable protecting group at its 5′ hydroxyl group. As stated previously, in an alternate embodiment, the protecting group is present at the 3′ hydroxyl group if synthesis of the polynucleotide is from the 5′ to 3′ direction. The nucleoside phosphoramidite is represented in accordance with one aspect of the present invention by the following formula: 1

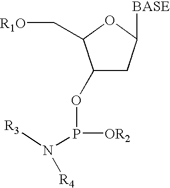

wherein the base is adenine, guanine, thymine, cytosine, or a base analogue such as a purine or pyrimidine analogue, R₁ is a protecting group which makes the 5′ hydroxyl group unavailable for reaction and includes dimethoxytrityl, tert-butyloxycarbonyl or any of the protecting groups known to those of skill in the art; R₂ is cyanoethyl, methyl, t-butyl, trimethylsilyl and the like and R₃ and R₄ are isopropyl, cyclohexane and the like. Exocyclic amines present on the bases can also be protected with acyl protecting groups such as benzoyl, isobutyryl, phenoxyacetyl and the like. The linker molecule contains an acid- or base-removable protecting group. Useful linker molecules are well known to those skilled in the art and representative examples include oligo ethers such as hexaethylene glycol, oligomers of nucleotides, esters, carbonates, amides and the like. Useful protecting groups include those previously listed and others known to those skilled in the art.

According to one aspect of the present invention, a process for fabricating an array of polymers is provided, the method having the steps of (a) coupling a monomer with a reactive site masked by a protecting group to a growing polymer chain; (b) capping the monomer or monomer chains that have not reacted in a giving cycle; (c) deprotecting the product with a deprotecting agent; (d) repeating steps (a)-(c) on regions of support as necessary to give a polymer having a desired sequence and length; and (e) oxidizing the intermonomer linkages post-synthetically. These steps are repeated as necessary to give an oligonucleotide having a desired sequence and length.

Typically, the coupling step in such a process is attachment of a first monomer with a reactive site masked by a protective group such as a protected 5′-hydroxyl to a solid support, usually through a linker, using standard methods and procedures known in the art. See for example, Oligonucleotides And Analogues A Practical Approach, Ekstein, F. Ed., IRL Press, N.Y, 1991. In case of a nucleoside, the nucleoside may be bound to the support through its 3′-hydroxyl group or its 5′-hydroxyl group, but is typically bound through the 3′-hydroxyl group.

The support-bound monomer or higher order first is then treated to remove the 5′-protecting group. Typically, this is accomplished by treatment with acid, base or irradiation. A second nucleoside monomer is then coupled to the free hydroxyl group of the support-bound initial monomer, wherein for 3′ to 5′ oligonucleotide synthesis, the second nucleoside monomer has a phosphorus derivative such as a phosphoramidite at the 3′ position and a protecting group at the 5′ position, and alternatively, for 5′ to 3′ oligonucleotide synthesis, the second nucleoside monomer has a phosphorus derivative at the 5′ position and a carbonate protecting group at the 3′ position. This coupling reaction gives rise to a newly formed phosphite triester bond between the initial nucleoside monomer and the added monomer, with the protected hydroxyl group intact. In the second step of the synthesis, the protected hydroxy group is removed and a third monomer is coupled to the deprotected second monomer. This process is repeated to selectively apply monomers until polymers of a desired length and desired chemical sequence are obtained. According to one aspect of the present invention, the growing chains of nucleic acid can be capped in between synthesis rounds. This procedure limits the production of nucleic acids with an undesired sequence.

It is generally preferable to perform a capping step between each coupling cycle. As understood by those skilled in the art, the capping step involves attachment of a “cap” moiety to oligonucleotide chains that have not reacted in a given coupling cycle. The cap moiety preferably is reactive with the terminal portion of oligonucleotides that did not participate in the coupling cycle but is not reactive with oligonucleotides that did participate and, moreover, is not itself reactive with the coupling reagents. Such a capping step is generally known to be beneficial by preventing shortened oligomer chains, by blocking chains that have not reacted in the coupling cycle. One representative reagent used for capping is acetic anhydride. Other suitable capping reagents and methodologies can be found in U.S. Pat. No. 4,816,571, issued Mar. 28, 1989, hereby incorporated by reference in its entirety.

In preferred embodiments, the phosphite triester, thiophosphite triester, or dithiophosphite triester linkage is oxidized to produce compounds having a desired internucleotide linkage. The choice of oxidizing agent will determine whether the linkage will be oxidized to, for example, a phosphotriester, thiophosphotriester, or a dithiophosphotriester linkage. Unlike the conventional method of solid phase oligonucleotide synthesis where the oxidation step occurs at each monomer addition cycle (e.g., 25 times for a 25mer), the oxidation step of the present invention is performed post-synthetically (single time). Post-synthetic oxidation not only decreases the overall number of steps that need to be performed to manufacture a probe array, but it also relieves the burden of extensive “washing” that is required to remove the aqueous oxidation reagent at each addition cycle. Because the probe arrays are synthesized more quickly and with fewer chemical steps, the throughput is higher and the chemical reagent costs are reduced. Probe fidelity is also increased as a result of reduced chemical exposure and processing time, which is particularly important for long-mer synthesis (i.e., 5-mer, 75-mer, 150-mer, etc.).

The protecting groups of this invention are typically removed by photolysis, i.e., by irradiation, though in selected cases it may be advantageous to use acid or base catalyzed cleavage conditions. Generally irradiation is at wavelengths greater than about 350 nm, preferably at about 365 nm. The photolysis is usually conducted in the presence of hydroxylic solvents, such as aqueous, alcoholic or mixed aqueous-alcoholic or mixed aqueous-organic solvent mixtures. Alcoholic solvents frequently used include methanol and ethanol. The photolysis medium may also include nucleophilic scavengers such as hydrogen peroxide. Photolysis is frequently conducted at neutral or basic pH.

It is contemplated that any photoremovable protecting group and any probe length is applicable to this strategy. In addition, many photolysis conditions will be applicable (e.g., dry, solvent assisted, etc.). In yet another variation of the invention, oxidation is made to occur during the (dry or wet) photolysis step.

The use of a photoremovable protecting group allows removal of selected portions of the substrate surface, via patterned irradiation, during the deprotection cycle of the solid phase synthesis. As discussed above, the formation of oligonucleotides on a solid-phase support requires the stepwise attachment of a nucleotide to a substrate-bound growing oligomer. In order to prevent unwanted polymerization of the monomeric nucleotide under the reaction conditions, protection of the 5′-hydroxyl group of the nucleotide is required. After the monomer is coupled to the end of the oligomer, the 5′-hydroxyl protecting group is removed, and another nucleotide is coupled to the chain. This cycle of coupling and deprotecting is continued for each nucleotide in the oligomer sequence. The use of a photoremovable protecting group allows removal, via patterned irradiation, of selected portions of the substrate surface during the deprotection cycle of the solid phase synthesis. This selectively allows spatial control of the synthesis the next nucleotide is coupled only to the irradiated areas.

Preferably, the photosensitive protecting groups will be removable by radiation in the ultraviolet (UV) or visible portion of the electromagnetic spectrum. More preferably, the protecting groups will be removable by radiation in the near UV or visible portion of the spectrum. In some embodiments, however, activation may be performed by other methods such as localized heating, electron beam lithography, x-ray lithography, laser pumping, oxidation or reduction with microelectrodes, and the like. Sulfonyl compounds are suitable reactive groups for electron beam lithography. Oxidative or reductive removal is accomplished by exposure of the protecting group to an electric current source, preferably using microelectrodes directed to the predefined regions of the surface which are desired for activation. Other methods may be used in view of this disclosure.

When light is used to activate or deactivate various groups, the light may be from a conventional incandescent source, a laser, a laser diode, or the like. If non-collimated sources of light are used it may be desirable to provide a thick- or multi-layered mask to prevent spreading of the light onto the substrate. It may, further, be desirable in some embodiments to utilize groups which are sensitive to different wavelengths to control synthesis. For example, by using groups which are sensitive to different wavelengths, it is possible to select branch positions in the synthesis of a polymer or eliminate certain masking steps.

Note that different photoprotected monomers can exhibit different photolysis rates. It may be desirable to utilize photoprotected monomers with substantially similar photolysis rates in a particular application. To obtain such a set of photoprotected monomers, one merely needs to select the appropriate photoprotecting group for each monomer in the set. In similar fashion, one can prepare a set of photoprotected monomers with substantially different photolysis rates (from monomer to monomer) by appropriate choice of photoprotecting groups.

Many, although not all, of the photoremovable protecting groups will be aromatic compounds that absorb near-UV and visible radiation. Suitable photoremovable protecting groups may be selected from a wide variety of positive light-reactive groups preferably including nitro aromatic compounds such as o-nitrobenzyl derivatives or benzylsulfonyl. In a preferred embodiment, NNPOC, DEACMOC, NPPOC, 6-nitroveratryloxycarbonyl (NVOC), 2-nitrobenzyloxycarbonyl (NBOC) or α,α-dimethyl-dimethoxybenz-yloxycarbonyl (DDZ) is used. Additional examples of the photoremovable protecting groups include multiply substituted nitro aromatic compounds containing a benzylic hydrogen ortho to the nitro group, wherein the substituent may include alkoxy, alkyl, halo, aryl, alkenyl, nitro, halo, or hydrogen. Other materials which may be used include o-hydroxy-α-methyl cinnamoyl derivatives. Further examples of photoremovable protective groups may be found in, for example, Patchomik, J. Am. Chem. Soc. (1970) 92:6333 and Amit et al., J. Org. Chem. (1974) 39:192.

The array is preferably an array of nucleic acids or an array of oligonucleotides. The monomer serves as a building block that can be used to make a macromolecule. Such building blocks include nucleic acids, nucleotides, nucleosides, and the like. Preferred nucleosides are deoxyadenosine, deoxycytidine, thymidine and deoxyguanosine as well as oligonucleotides incorporating such nucleosides. The monomer is preferably a naturally or non-naturally occurring nucleotide. More preferably the nucleotide is selected from the group consisting of G, A, T, and C. When nucleotide and oligonucleotide compositions are used, with the protecting groups of this invention, the protecting groups are preferably incorporated into the 3′-OH or the 5′-OH of the nucleoside. Thus, in accordance with the present invention, nucleotides may be synthesized in the 5′ to 3′ direction or a 3′ to 5′ direction.

The use of nucleoside and nucleotide analogs is also contemplated by this invention to provide oligonucleotide or oligonucleoside analogs bearing the protecting groups disclosed herein. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into an oligonucleotide or oligonucleoside sequence, they allow hybridization with a naturally occurring oligonucleotide sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired.

Analogs also include protected and/or modified monomers as are conventionally used in oligonucleotide synthesis. As one of skill in the art is well aware oligonucleotide synthesis uses a variety of base-protected deoxynucleoside derivatives in which one or more of the nitrogens of the purine and pyrimidine moiety are protected by groups such as dimethoxytrityl, benzyl, tert-butyl, isobutyl and the like. Specific monomeric building blocks which are encompassed by this invention include base protected deoxynucleoside H-phosphonates and deoxynucleoside phosphoramidites.

For instance, structural groups are optionally added to the ribose or base of a nucleoside for incorporation into an oligonucleotide, such as a methyl, propyl or allyl group at the 2′-O position on the ribose, or a fluoro group which substitutes for the 2′-O group, or a bromo group on the ribonucleoside base. 2′-O-methyloligoribonucleotides (2′-O-MeORNs) have a higher affinity for complementary nucleic acids (especially RNA) than their unmodified counterparts. 2′-O-MeORNA phosphoramidite monomers are available commercially, e.g., from Chem Genes Corp. or Glen Research, Inc. Alternatively, deazapurines and deazapyrimidines in which one or more N atoms of the purine or pyrimidine heterocyclic ring are replaced by C atoms can also be used.

The phosphodiester linkage, or “sugar-phosphate backbone” of the oligonucleotide analogue can also be substituted or modified, for instance with methyl phosphonates or O-methyl phosphates. Another example of an oligonucleotide analogue for purposes of this disclosure includes “peptide nucleic acids” in which a polyamide backbone is attached to oligonucleotide bases, or modified oligonucleotide bases. Peptide nucleic acids which comprise a polyamide backbone and the bases found in naturally occurring nucleosides are commercially available.

Nucleotides with modified bases can also be used in this invention. Some examples of base modifications include 2-aminoadenine, 5-methylcytosine, 5-(propyn-1-yl)cytosine, 5-(propyn-1-yl)uracil, 5-bromouracil, and 5-bromocytosine which can be incorporated into oligonucleotides in order to increase binding affinity for complementary nucleic acids. Groups can also be linked to various positions on the nucleoside sugar ring or on the purine or pyrimidine rings which may stabilize the duplex by electrostatic interactions with the negatively charged phosphate backbone, or through hydrogen bonding interactions in the major and minor groves. For example, adenosine and guanosine nucleotides can be substituted at the N.sup.2 position with an imidazolyl propyl group, increasing duplex stability. Universal base analogues such as 3-nitropyrrole and 5-nitroindole can also be included. A variety of modified oligonucleotides and oligonucleotide analogs suitable for use in this invention are described “Antisense Research and Applications”, S. T. Crooke and B. LeBleu (eds.) (CRC Press, 1993) and “Carbohydrate Modifications in Antisense Research” in ACS Symp. Ser. #580, Y. S. Sanghvi and P. D. Cook (eds.) ACS, Washington, D.C. 1994).

The methods of the present invention involve sequential addition of monomers to build up an array of polymeric species on a support by activating predefined regions of a substrate or solid support and then contacting the substrate with a protected monomer of this invention (e.g., a protected nucleoside amino acid). It will be recognized that the individual monomers can be varied from step to step. A common support is a glass or silica substrate as is used in semiconductor devices.

The predefined regions can be activated with a light source, typically shown through a screen such as a photolithographic mask similar to the techniques used in integrated circuit fabrication. Other regions of the support remain inactive because they are blocked by the mask from illumination and remain chemically protected. Thus, a light pattern defines which regions of the support react with a given monomer. The protected monomer reacts with the activated regions and is immobilized therein. The protecting group is removed by photolysis and washed off with unreacted monomer. By repeatedly activating different sets of predefined regions and contacting different monomer solutions with the substrate, a diverse array of polymers of known composition at defined regions of the substrate can be prepared. Arrays of 10⁶, 10⁷, 10⁸, 10⁹, 10¹⁰, 10¹¹, 10¹² or more different polymers can be assembled on the substrate. The regions may be 1 mm² or larger, typically 10 μm² and may be as small as 1 μm². In another embodiment, arrays may comprise about or more than about 10³ 10⁴ 10⁵ 10⁶, 10⁷, 10⁸, 10⁹, 10¹⁰, 10¹¹, 10¹² different polymers per cm².

The methods described herein may also employ component molecules comprising a masked reactive site attached to a photolabile protecting group. In such cases, the protecting group is attached to an acidic reactive site, such as a carboxylate or phosphate and is removed by photolysis.

In the nucleic acid embodiments of the invention, and as described above, the solid substrate or solid support may be of any form, although they preferably will be planar and transparent (and potentially some three dimensional structure). The supports need not necessarily be homogenous in size, shape or composition, although the supports usually and preferably will be uniform. In some embodiments, supports that are very uniform in size may be particularly preferred. In another embodiment, two or more distinctly different populations of solid supports may be used for certain purposes. In another embodiment, the solid support may comprise sets of beads with one nucleic acid species synthesized on each bead. The beads may be separate (free from each other or free from a second support), may be attached to a second support or may be held within wells, trenches or other feature on a second support, such as a chip or slide, for instance a glass chip or slide.

Solid supports may consist of many materials, limited primarily by capacity for derivatization to attach any of a number of chemically reactive groups and compatibility with the synthetic chemistry used to produce the array and, in some embodiments, the methods used for tag attachment and/or synthesis. Suitable support materials typically will be the type of material commonly used in peptide and polymer synthesis and include glass, latex, heavily cross-linked polystyrene or similar polymers, gold or other colloidal metal particles, and other materials known to those skilled in the art. The chemically reactive groups with which such solid supports may be derivatized are those commonly used for solid phase synthesis of the polymer and thus will be well known to those skilled in the art, i.e., carboxyls, amines, and hydroxyls.

To improve washing efficiencies, one can employ nonporous supports or other solid supports less porous than typical peptide synthesis supports; however, for certain applications of the invention, quite porous beads, resins, or other supports work well and are often preferable. One such support is a resin in the form of beads. In general, the bead size is in the range of 1 nm to 100 μm, but a more massive solid support of up to 1 mm in size may sometimes be used. Particularly preferred resins include Sasrin resin (a polystyrene resin available from Bachem Bioscience, Switzerland); and TentaGel S AC, TentaGel PHB, or TentaGel S NH2 resin (polystyrene-polyethylene glycol copolymer resins available from Rappe Polymere, Tubingen, Germany). Other preferred supports are commercially available and described by Novabiochem, La Jolla, Calif.

In accordance with another aspect of the present invention, an array of oligonucleotides is produced using post-synthetic or intermittent oxidation to produce features preferably on the order of 10-100 μm. More preferably, features are on the order 1-10 μm. In another preferred embodiment, features are on the order of 100-1000 nm.

In accordance with another aspect of the present invention, a process for fabricating an array of polymers is provided, the method having the steps of (a) coupling a monomer with a reactive site masked by a protecting group to a growing polymer chain; (b) optionally capping the monomer or monomer chains that have not reacted in a giving cycle; (c) deprotecting the product with a deprotecting agent; (d) repeating steps (a)-(c) on regions of support as necessary to give a polymer having a desired sequence and length; and (e) oxidizing the intermonomer linkages every N cycles, wherein N is, for example, about 5, 10, 25, 100 or more. This variation is referred to as intermittent oxidation and may additionally include a post-synthesis oxidation step.

Although oxidation can be performed post-synthetically and/or intermittently, it is important that the final oxidation precedes the base-mediated wafer deprotection step (typically performed with EDA/EtOH solution).

What is claimed is:

1. A process for fabricating an array of polymers on a solid support, comprising the steps of:
(a) coupling a monomer with a reactive site masked by a protecting group to a growing polymer chain;
(b) optionally capping the monomer or monomer chains that have not reacted in a giving cycle;
(c) deprotecting the product with a deprotecting agent;
(d) repeating steps (a)-(c) on regions of support as necessary to give a polymers having a desired sequence and length; and
(e) oxidizing the intermonomer linkages post-synthetically.

2. The process of claim 1 wherein said array of polymers comprises an array of nucleic acids.

3. The process of claim 1 wherein said array of polymers comprises an array of oligonucleotides.

4. The process of claim 1 wherein said polymer is a DNA oligonucleotide.

5. The process of claim 1 wherein said monomer is a nucleotide.

6. The process of claim 1, wherein the protective group is a photolabile group.

7. The process of claim 5 wherein said nucleotide is protected at its 5′ hydroxyl end with a photolabile protective group.

8. The process of claim 5 wherein said nucleotide is protected at its 3′ hydroxyl group with a photolabile protective group.

9. The process of claim 1 wherein said deprotecting step comprises photolysis.

10. The process of claim 5 wherein said nucleotide is selected from the group consisting of G, A, T and C.

11. The process of claim 10 wherein said nucleotide selected from the group consisting of G, A, T, and C is protected at its 5′ hydroxyl group with a photolabile protective group.

12. The process of claim 1, wherein said intermonomer linkage is selected from the group consisting of phosphite triester, thiophosphite triester, and dithiophosphite triester bonds.

13. The process of claim 1 wherein the capping step is performed prior to oxidation.

14. The process of claim 1 wherein said array of polymers comprises a polymer at least 50 monomers in length.

15. The process of claim 13 wherein said array of polymers comprises a polymer at least 60 monomers in length.

16. A process of claim 14 wherein said array of polymers comprises a polymer at least 70 nucleotides in length.

17. An array of polymers produced in accordance with claim 1 comprising a feature is on the order of 10-100 μm.

18. An array of polymers produced in accordance with claim 1 having a feature on the order of 1-10 μm.

19. An array of polymers produced in accordance with claim 1 having a feature on the order of 100 to 1000 nm.
